(19) United States
(12) Patent Application Publication
(10) Pub. No.: US 2017/0112948 A1
(43) Pub. Date: Apr. 27, 2017

(54) Inorganic-organic hybrid compound

(71) Applicants: Karlsruher Institut für Technologie, Karlsruhe (DE); Max-Planck-Gesellschaft zur Förderung der Wissenschaften E.V., München (DE); GEORG-AUGUST-UNIVERSITÄT STIFTUNG ÖFFENTLICHEN RECHTS, UNIVERSITÄTSMEDIZIN, Göttingen (DE)

(72) Inventors: JOACHIM HECK, Sinzheim (DE); MARIEKE POSS, Karlsruhe (DE); HOLGER REICHARDT, Göttingen (DE); JOANNA NAPP, Rosdorf (DE); FRAUKE ALVES, Göttingen (DE); WALTER STÜHMER, Göttingen (DE); CLAUS FELDMANN, Ettlingen (DE)

(21) Appl. No.: 15/129,430

(22) PCT Filed: Feb. 26, 2015
(86) PCT No.: PCT/EP2015/000454

§ 371 Date: Sep. 27, 2016

(30) Foreign Application Priority Data
Mar. 28, 2014 (DE) 10 2014 004 512.9

(51) Int. Cl. A61K 49/00; A61K 31/661; A61K 31/675; A61K 31/7056; A61K 31/6615; A61K 31/7068; A61K 31/7076; A61K 31/662; A61K 31/663; A61K 31/665; A61K 31/569; A61K 31/65; A61K 31/704; A61K 31/427; A61K 31/7008; A61K 38/12; A61K 31/546; A61K 31/519; A61K 31/185; A61K 31/683; A61K 47/02

(52) U.S. Cl. CPC A61K 49/0041; A61K 47/02; A61K 49/0021; A61K 31/661; A61K 31/675; A61K 31/7056; A61K 31/6615; A61K 31/7068; A61K 31/7076; A61K 31/662; A61K 31/663; A61K 31/665; A61K 31/569; A61K 31/65; A61K 31/704; A61K 49/0034; A61K 49/0036; A61K 49/0032; A61K 31/427; A61K 31/7008; A61K 38/12; A61K 31/546; A61K 31/519; A61K 31/185; A61K 31/683

(57) ABSTRACT

The present invention relates to an inorganic-organic hybrid compound as ionic compound, composed of an inorganic cation and of an organic active ingredient anion and also, optionally, of an organic fluorescent dye anion.

The present invention relates to an inorganic-organic hybrid compound as ionic compound, consisting of an inorganic cation and an organic active ingredient anion, and also, optionally, of an organic fluorescent dye anion. Founded on a simple, water-based synthesis, a broad palette of the inorganic-organic hybrid compound substances of the invention is available, with a broad active-ingredient and therapeutic basis (e.g., inflammatory or immunological disorders such as rheumatism, arthritis, osteoporosis, multiple sclerosis; neurological disorders such as epilepsy or schizophrenia; tumor disorders; infectious diseases (bacterial, viral, parasitic) such as malaria, tuberculosis or mycoses; cardiovascular disorders such as angina pectoris or coronary deposits; for the therapy of pain). Moreover, a combination of active ingredient release with optical detection is an easy possibility. The inorganic-organic hybrid compounds of the invention combine therapy (active ingredient release) with diagnostics (optical detection of the hybrids and also CT-based or MRI-based detection). Under physiological conditions, the active ingredient is released very slowly, over a period of several hours up to several days, and is able to develop its effect in a targeted way at the locus of action, with the hybrid compound being fully broken down.

The targeted delivery of drugs with nanoparticles represents a major challenge for present-day interdisciplinary sciences. In this field, a large number of different materials approaches and therapeutic approaches are currently being pursued, for a very wide variety of clinical pictures. The encapsulation of active ingredients in vesicles, meso-scale or nano-scale hollow beads, or polymer capsules is widespread. Furthermore, active ingredients are being bound on nanoparticles such as SiO₂, Au, quantum dots or polymer nanoparticles. Alternatively, active ingredients can also be embedded in nanoparticles, with SiO₂ and polymers being the most commonplace matrix materials. Disadvantages of the materials and solutions presently under discussion are the in some cases very small amount of active ingredient in relation to the nanoparticles overall (e.g., 1% active ingredient encapsulated in 99% SiO₂ as nanoparticle matrix), and the incomplete degradability and/or potential toxicity of the nanoparticles after the active ingredient has been released (for example, SiO₂ nanoparticles remain). Moreover, the materials approaches and the synthesis of the materials are complex and very involved. This is especially the case for multifunctional nanoparticles which in addition to active ingredient release also allow analytical detection.

US 2011/0064775 A1 describes the encapsulation of nanoparticles (e.g., Fe₃O₄ in working example 1) or of an organic fluorescent dye molecule (e.g., fluorescein in working example 2 or 3), or of an organic active ingredient molecule (e.g., doxorubicin in working example 4), in an organometallic coordination compound consisting of a zinc salt (e.g., zinc nitrate) and 1,4-bis(imidazol-1-ylmethyl)benzene (Bix) as coordination ligand. The compounds described are prepared for example in absolute (i.e., anhydrous) ethanol.

U.S. Pat. No. 8,779,175 B2 describes water-soluble coordination complexes which contain an active ingredient molecule.

DE 20 2006 024 289 A1 discloses molecular anions which are bonded to layers of layer-forming metal hydroxides of formula M²⁺₁₋ₓM³⁺ₓ(OH)₂(Zⁿ⁻)_{x/n}. The structure-imparting metal hydroxide is formed from the cations M^{2+/3+} and from hydroxide ions (OH), which do not fluoresce and do not constitute an active ingredient.

In summary, the following points may be given as disadvantages of prior-art drug delivery systems and materials:
involved chemical synthesis, multistage processes;
complex materials systems such as core/shell structures, and complicated modification of the particle surface;
small amount of active ingredient relative to the overall mass of the nanoparticles;
constituents which are toxic and/or which are excretable/degradable incompletely or only very slowly under physiological conditions;
activity of a complex system of materials against only one clinical picture;
active ingredient bonded to a nanoparticle only superficially, and therefore easily abradable in suspension as a result of particle impacts;
incidence of side-effects;
excessively rapid or excessively slow release of the particular drugs.

Against this background it is an object of the present invention to provide a simple materials concept in conjunction with a very broad application spectrum that is available by a very simple synthesis, permits a large amount of active ingredient per nanoparticle, and at the same time allows optical detection.

This object is achieved by the embodiments specified in the claims.

Provided in particular is an inorganic-organic hybrid compound as ionic compound, composed of an inorganic cation and an organic active ingredient anion and also, optionally, of an organic fluorescent dye anion, the compound having a molar solubility of 10⁻² mol/l in water. Active ingredient anion and fluorescent dye anion, if present, each contain at least one phosphate, phosphonate, sulfate, sulfonate, carbonate or carboxylate group as functional group, which in conjunction with the inorganic cation enable the formation of a compound which is sparingly soluble in water, and hence enable the formation of nanoparticles. The inorganic-organic hybrid compound of the invention preferably comprises an active ingredient anion and a fluorescent dye anion, allowing the inorganic-organic hybrid compound to release an active ingredient and to be located in cells, tissues, and organs by light emission, owing to the fluorescence of the fluorescent dye anion. The hybrid compound is preferably excited with visible light. The emission of the hybrid compound is preferably in the visible to infrared spectral range of light.

By “active ingredient anion” is meant a substance which is designated as an agent for curing or for preventing human or animal diseases, and also a substance which is intended, in or on the human or animal body, to provide a medical diagnosis or for restoration, enhancement or modification of the human or animal bodily functions.

The active ingredient anion here may be used against a very wide variety of different clinical pictures. The active ingredient anion is preferably employed against chronic inflammation/asthma/rheumatism/arthritis/multiple sclerosis, inflammation in general, tumor disorders, malaria, tuberculosis, angina pectoris, or coronary deposits. Under physiological conditions, the active ingredient anion can preferably be released with a time delay over a range from several hours up to several days. Release preferably takes place simply, by hydrolysis under physiological conditions or in the presence of phosphatases, through ester cleavage. A feature of the inorganic-organic hybrid compound of the invention is that its constituents are not allergenic and/or not toxic and are degraded and/or excreted fully under physiological conditions.

The organic active ingredient anion incorporated into the inorganic-organic hybrid compound of the invention is not subject to any substantial restriction, provided it has at least one phosphate, phosphonate, sulfate, sulfonate, carbonate or carboxylate group, preferably phosphate, phosphponate, sulfate or sulfonate group, as functional group, which in conjunction with the inorganic cation permits the formation of a compound which is sparingly soluble in water, and hence permits the formation of nanoparticles. As already observed above, preferred active ingredient anions are those which act against chronic inflammation/rheumatism/arthritis/multiple sclerosis, inflammation in general, tumor disorders, malaria, tuberculosis, angina pectoris, or coronary deposits. Active ingredients of this kind are known to the skilled person. For the purposes of the present invention, use may be made, by way of example, of acetaminophen phosphate, betamethasone phosphate, dexamethasone phosphate, uridine monophosphate, 5′-fluoro-2′-deoxyuridine 5′-monophosphate (FdUMP), methyl-prednisolone phosphate, triamcinolone phosphate, estrone phosphate, testosterone phosphate, estramustine phosphate, codeine phosphate, clindamycin phosphate, thiamine pyrophosphate, thiamine phosphate; aracytidine monophosphate, cyclic 3′,5′-adenosine monophosphate, vidaribine phosphate, 9-[9-(phosphonomethoxy)ethoxy]adenine, fospropofol, fosphenytoin, phosphoryloxymethyloxymethylphenytoin, phosphoryloxymethylphenylbutazone, phosphoryloxymethyloxymethylphenylbutazone, phosphoryloxymethylphenindione, phosphoryloxymethyloxymethylphenindione, N-phosphonooxymethylcinnarizine, N-phosphonooxymethylloxapine, N-phosphonooxymethylamiodarone, alendronate, canrenoate, doxycyclin hydrate, doxorubicin hydrochloride, aztreonam, tigemonam, D-glucosamine 6-sulfate, colistin methane sulfate, cefsulodin, fosamprenavir, tenofovir, adefovir, combretastatin A-4 phosphate, folic acid, 2-mercaptoethansulfonate/mesna, fosfomycin, glyphosate, glufosinate, zolendronate, aminotrimethylenephosphonic acid, diethylenetriamine-penta(methylenephosphonic acid), ethylenediaminetetra(methylenephosphonic acid), fosbretabulin, α-tocopherol phosphate, VAPOL hydrogenphosphate, pyridoxal 5′-phosphate 6-(2′-naphthylazo-6′-nitro-4′,8′-disulfonate), (11bR)-2,6-di-9-phenanthrenyl-4-hydroxydinaphtho[2,1-d:1′,2′-f] [1,3,2]dioxaphosphepine 4-oxide, 8-bromo cyclic adenosine diphosphate ribose, phytic acid, glucose 6-phosphate and other phosphoric esters of sugars, or naturally occurring and synthetic nucleotides (for example, adenosine monophosphate (AMP), adenosine diphosphate (ADP), adenosine triphosphate (ATP), guanosine monophosphate (GMP), guanosine diphosphate (GDP), guanosine triphosphate (GTP), cytidine monophosphate (CMP), cytidine diphosphate (CDP), cytidine triphosphate (CTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxycytidine monophosphate (dCMP), deoxycytidine diphosphate (dCDP), deoxycytidine triphosphate (dCTP), deoxythymidine monophosphate (dTMP), deoxythymidine diphosphate (dTDP) or deoxythymidine triphosphate (dTTP)). Furthermore, it is also possible to use organic active ingredients which do not as such have any phosphate, phosphonate, sulfate, sulfonate, carbonate or carboxylate group, by modifying them with at least one of these functional groups. Corresponding methods for the functionalization of such organic active ingredients are known to the skilled person.

For the purposes of the present invention, the inorganic cation is selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or a lanthanoid. Particularly preferred are the cations Mg²⁺, Ca²⁺, [ZrO]²⁺ or La³⁺.

For the purposes of the present invention it is possible to carry out detection of the inorganic-organic hybrid compounds of the invention not only optically, via the fluorescence of the fluorescent dye anion, but also by X-ray absorption or magnetic measurements in the presence of heavy or magnetic inorganic cations (e.g. Ba²⁺, [ZrO]²⁺, [HfO]²⁺, Gd³⁺, La³⁺, Fe³⁺, Bi³⁺).

The inorganic-organic hybrid compound of the invention may also be understood as comprising an inorganic matrix and an organic active ingredient compound, with the inorganic matrix being composed of an inorganic compound selected from the group consisting of metal phosphates, including the hydrogen phosphates and dihydrogen phosphates, metal oxide phosphates, metal phosphonates, metal sulfates, metal sulfonates, metal carbonates or metal carboxylates, with the inorganic compound comprising a cation selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or a lanthanoid, with the organic active ingredient compound having one or more functional groups selected from phosphate, phosphonate, sulfate, sulfonate, carbonate or carboxylate groups, via which the organic active ingredient compound is incorporated into the inorganic matrix. In so far as the active ingredient compound and the optional fluorescent dye are incorporated via the functional group thereof into the inorganic matrix by means of ionic bonding, accordingly, the functional group, phosphate for example, is then considered part of the inorganic matrix—in other words, there is no independent phosphate group on the active ingredient compound.

Both the organic active ingredient compound and the organic fluorescent dye are incorporated into the hybrid compound or inorganic matrix by way of the anionic functional groups, in other words by means of ionic bonding. The present invention embraces those inorganic-organic hybrid compounds which comprise molar amounts of active ingredient compound, and also “diluted” variants. Both variants can be obtained by simple mixing of aqueous solutions of the starting materials.

In accordance with the present invention, the inorganic-organic hybrid compound may comprise the active ingredient anion, optionally together with the fluorescent dye anion, in molar amounts, with the molar amount of active ingredient anion and fluorescent dye anion being in a stoichiometric proportion to the inorganic cation, taking account of the respective ion charges. In the inorganic-organic hybrid compound, however, it is also possible for the ratio of active ingredient anion to fluorescent dye anion to be varied.

In another embodiment of the present invention, the inorganic-organic hybrid compound is further functionalized with an antibody or peptide such as, for example, antibodies and antibody fragments, nanobodies, diabodies, peptide aptamers, or with an oligonucleotide, such as aptamers or similar molecules, for example, in order to channel the inorganic-organic hybrid nanoparticles and the active ingredient contained to a specific locus of action in vivo and to accumulate them there. Because of the water-based synthesis of the inorganic-organic hybrid compounds, this coupling with antibodies or similar molecules is particularly simple and gentle.

In one preferred embodiment of the present invention, the inorganic-organic hybrid compound, as already observed above, further comprises an organic fluorescent dye, i.e., a corresponding anion thereof, which has one or more functional groups selected from phosphate, phosphonate, sulfate, sulfonate, carbonate or carboxylate groups, via which the fluorescent dye (anion) is incorporated into the ionic compound or inorganic matrix. The organic fluorescent dye is preferably selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluoro-phenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N,N′-difluoroboryl-1,9-dimethyl-5-(4-iodophenyl)dipyrrin, N, N′-difluoroboryl-1,9-dimethyl-5-[(4-(2-trimethylsilylethynyl), N, N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenylbetaocta(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, for example, tetra(o-amidophosphonatophenyl)porphyrin, and umbelliferone. Here, the organic fluorescent dyes which as such do not have any phosphate, phosphonate, sulfate, sulfonate, carbonate or carboxylate group are modified with at least one of these functional groups (for example: phenylumbelliferone phosphate (PUP), methylfluorescein phosphate (MFP), resorufin phosphate (RRP), Dyomics-647-uridine phosphate (DUT)). Appropriate methods for functionalizing such organic fluorescent dyes are known to a skilled person.

In another preferred embodiment of the present invention, the organic fluorescent dye is selected from the group consisting of riboflavin 5′-monophosphate sodium salt, fluorescein, resorufin, amaranth, rhodamine, perylene, coumarin, and umbelliferone, the latter functionalized with at least one phosphate, phosphonate, sulfate, sulfonate, carbonate or carboxylate group. An example that may be given here is phenylumbelliferone phosphate.

The inorganic-organic hybrid compound is sparingly soluble. For the purposes of the present invention, sparingly soluble compounds are understood to be those having a molar solubility of ≦10⁻² mol/l. The sparingly soluble compounds preferably have a molar solubility of 10⁻⁴ mol/l. This is advantageous for the synthesis of the inorganic-organic hybrid compounds of the invention, since accordingly the inorganic-organic hybrid compound, which comprises the active ingredient compound and also the fluorescent dye, where provided, can be precipitated from soluble precursor compounds.

The inorganic-organic hybrid compound customarily has an X-ray-amorphous structure. This is advantageous for simplified synthesis, since amorphous nanoparticles can be obtained without substantial synthetic cost and complexity.

In one preferred embodiment of the present invention, the inorganic-organic hybrid compound is additionally doped with one or more cations and/or anions. Doping makes it possible to modify the luminescence properties of the hybrid compound of the invention, since, following excitation of the organic fluorescent dye, there is a partial or complete transfer of energy to the dopant, and so subsequently an emission can be observed that originates from the dopant. It is further possible for the doping to alter the excitation of the hybrid compound of the invention. Doping may be carried out at any suitable concentration range. The doping is present preferably in a concentration range of 5 ppm to 50 mol %, more preferably in a concentration range from 0.1 to 5.0 mol %. The inorganic-organic hybrid compound is doped preferably with a lanthanoid selected from Ce, Pr, Nd, Sm, Eu, Gd, Tb, Dy, Ho, Er, Tm, Yb or Lu, with a transition metal selected from Cr, Mn, Cu, Zn, Y, Ag or Cd, with a main group element selected from Sn, Sb, Pb or Bi, or with a complex anion selected from [VO₄]³⁻, [MoO₄]³⁻ or [WO₄]³⁻.

The hybrid compound of the invention may have any suitable particle size. In one preferred embodiment the hybrid compound of the invention is nanoscale and has a particle diameter in the range from 1 to 100 nm. Particularly preferred is a particle diameter in the range from 1 to 20 nm. Furthermore, the hybrid compound of the invention preferably has a virtually monodisperse size distribution in the range of ±30%, more preferably in the range of ±5%. Furthermore, the hybrid compound of the invention preferably has a low degree of agglomeration, more preferably with a size distribution in the range of ±30%, even more preferably in the range of ±5%. Suitable methods for determining the particle diameter and the monodisperse size distribution are known in the prior art.

In accordance with the present invention it is possible in each case for different excitation properties and emission properties to be set for a given hybrid compound, through the choice and the incorporation of a corresponding organic fluorescent dye. The excitation of the hybrid compound of the invention is situated preferably in the range from 100 to 800 nm, and the emission in the range from 200 to 2000 nm. Excitation is achieved in general by a light-emitting diode or by a laser emitting visible to near-infrared light (i.e., 450 to 800 nm), and emission of the organic fluorescent dye or of the hybrid compound of the invention is in the visible spectral range between blue and infrared (i.e., 450 to 1400 nm). In one embodiment the excitation is in the form of UV light (i.e., 100 to 450 nm). With the hybrid compound of the invention, the luminescent intensity under excitation conditions preferably does not decrease very much over the stimulation time, in comparison to the unbound organic fluorescent dye, and with particular preference the luminescent intensity does not decrease over the excitation time, especially in the case of excitation with a light-emitting diode. Accordingly, on exposure to UV light, the luminescent intensity decreases preferably by not more than 10%, and by not more than 1% on exposure to daylight.

The present invention further relates to a process for preparing the hybrid compound of the invention, comprising the steps of
(a) providing a solution of an organic active ingredient compound which has one or more functional groups selected from phosphate, phosphonate, sulfate, sulfonate, carbonate or carboxylate groups, the solution optionally further comprising at least one anion selected from phosphate, phosphonate, sulfate, sulfonate, carbonate or carboxylate,
(b) providing a solution of a soluble metal salt comprising metal cations selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or a lanthanoid,
(c) combining the two solutions with stirring, to precipitate the hybrid compound, and
(d) isolating and/or purifying the precipitated hybrid compound.

Step (a) of the process of the invention comprises the providing of a solution of the organic active ingredient compound. Furthermore, this solution may optionally further comprise at least one anion, selected from phosphate, phosphonate, sulfate, sulfonate, carbonate or carboxylate. If used, this anion may be present together with a cation in the form of a dissolved salt, in the form for example of dissolved alkali metal sulfate, alkali metal phosphate, alkali metal carboxylate, alkali metal carbonate. The alkali metal is preferably sodium or potassium. The anion may also be present in the form of the corresponding acid in the solution. In one embodiment of the present invention the solution for providing one of the aforementioned anions comprises an acid from the group consisting of sulfuric acid, phosphoric acid or a carboxylic acid. In the case of the carboxylic acid, the acid is preferably formic acid, acetic acid, propionic acid or oxalic acid. In the case of the carboxylate, the carboxylate, accordingly, is preferably formate, acetate or propionate. With particular preference the solution comprises phosphate as anion, and preferably phosphoric acid is used to provide this anion.

In one preferred embodiment of the present invention, the solution provided in step (a) is admixed further with an organic fluorescent dye which has one or more functional groups selected from phosphate, phosphonate, sulfate, sulfonate, carbonate or carboxylate group.

As solvent it is possible to use any suitable solvent. Preferred for use as solvent is water, isotonic water, a physiological buffer, an alcohol, or a mixture of two or more of these solvents. Preferred alcohols for use as solvents are methanol, ethanol, propanol, and isopropanol.

Step (b) of the process of the invention comprises the providing of a solution of a soluble metal salt which comprises metal cations which may be identical or different and are selected from Mg²⁺, Ca ²⁺, Sr²⁺, Ba ²⁺, Zn ²⁺, Zr ⁴⁺, [ZrO]²⁺, [HfO]²⁺, Sc³⁺, Y³⁺, Gd³⁺, La³⁺, Fe³⁺, Bi³⁺ or a lanthanoid. As solvent it is possible to use any suitable solvent. Preferred for use are, again, the solvents identified above, namely water, isotonic water, a physiological buffer, alcohols, and mixtures of two or more of these solvents. One particularly preferred embodiment of the present invention uses isotonic water or a physiological buffer as solvent. As metal salt it is possible to use any salt which is soluble in the solvent used. Suitable metal salts are known to the skilled person. As metal salt it is possible with preference to use the halides, nitrates, and sulfates of the abovementioned metals, provided they are soluble in the particular solvent used. In one particularly preferred embodiment of the present invention, magnesium dichloride, calcium dichloride, lanthanum trichloride or zirconyl chloride is used as metal salt.

Step (c) of the process of the invention comprises the combining of the two solutions with stirring. In this way the hybrid compound of the invention is precipitated. In the combining step, the two solutions may have any suitable temperature. In one preferred embodiment of the present invention, at least one of the two solutions has, or both solutions have, a temperature in the range from room temperature to 85° C., more preferably a temperature in the range from 40° C. to 75° C. The two solutions are preferably combined rapidly, in other words within a period of not more than 10 seconds, preferably within a period of not more than 5 seconds.

Step (d) of the process of the invention comprises the isolating and/or purifying of the precipitated hybrid compound. This isolating and/or purifying may take place by any suitable methods. Such methods are known in the prior art.

The isolating and/or purifying of the hybrid compound particles is preferably accomplished by a method selected from the group consisting of centrifugation techniques, dialysis techniques, phase transfer techniques, chromatography techniques, ultrafiltration techniques, washing techniques, and combinations thereof. The abovementioned methods for the isolating and/or purifying of the hybrid compound particles may also be performed in combination and/or multiply.

In the figures:

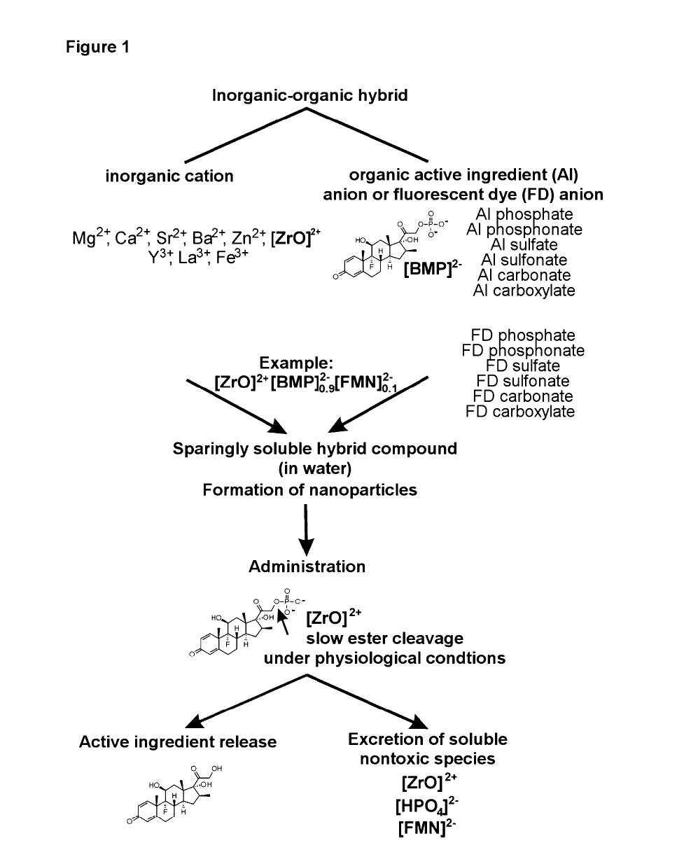

FIG. 1 shows a schematic representation of the synthesis and use of an inorganic-organic hybrid compound having an active ingredient anion and a fluorescent dye anion, using the example of [ZrO]²⁺[BMP]²⁻_{0.9}[FMN]²⁻_{0.1} (BMP: betamethasone phosphate; FMN: flavin mononucleotide);

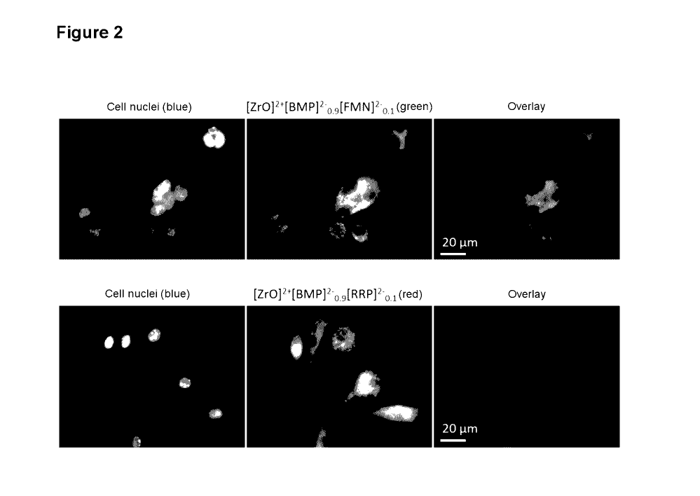

FIG. 2 shows light micrographs of inorganic-organic hybrid compounds having an active ingredient anion and a fluorescent dye anion in macrophages on the basis of the fluorescence of the fluorescent dye anion, using the example of [ZrO]²⁺[BMP]²⁻_{0.9}[FMN]²⁻_{0.1} (green) and of [ZrO]²⁺[BMP]²⁻_{0.9}[RRP]²⁻_{0.1} (red) (BMP: betamethasone phosphate; FMN: flavin mononucleotide; RRP: resorufine phosphate). Cell nuclei are stained with DAPI (blue);

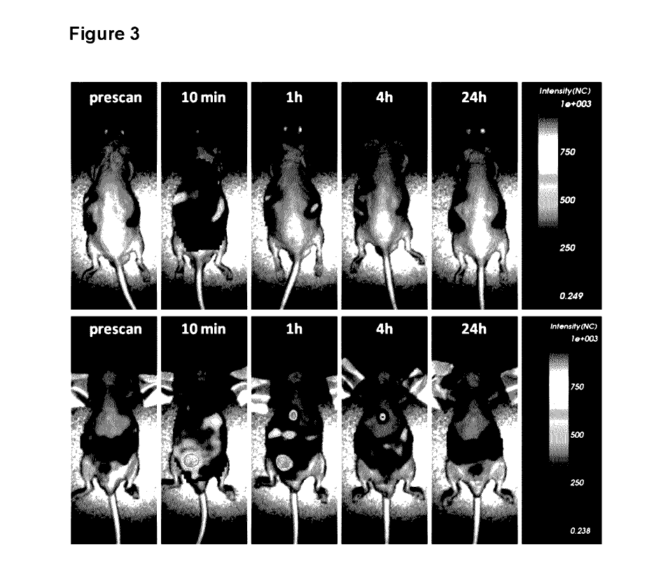

FIG. 3 shows a demonstration of the distribution of inorganic-organic hybrid compounds having an active ingredient anion and a fluorescent dye anion in the mouse model following intravenous administration, on the basis of the fluorescence of the fluorescent dye anion, using the example of [ZrO]²⁺[BMP]²⁻_{0.95}[IRF]²⁻_{0.05} (BMP: betamethasone phosphate; IRF: IR-emitting fluorescent dye);

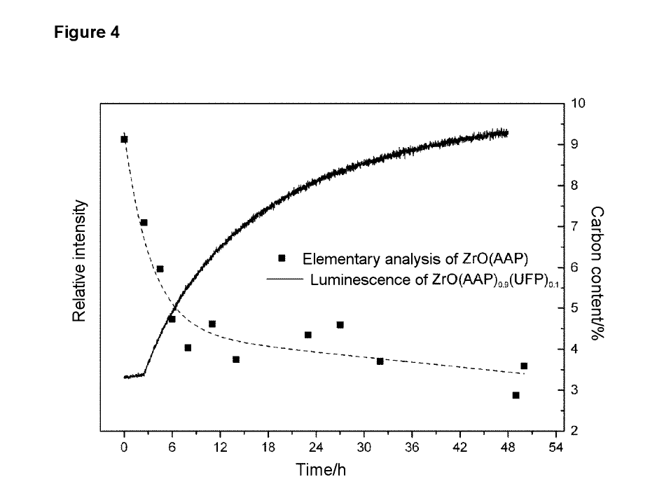

FIG. 4 shows the release of active ingredient from inorganic-organic hybrid compounds having an active ingredient anion and a fluorescent dye anion, using the example of [ZrO]²⁺[AAP]²⁻_{0.9}[UFP]²⁻_{0.1}, the release being shown by detection of the fluorescence of the released fluorescent dye (UFP), and also the decrease of the carbon content in the remaining nanoparticles, by elemental analysis (AAP: acetaminophen phosphate; UFP: umbelliferone phosphate); and

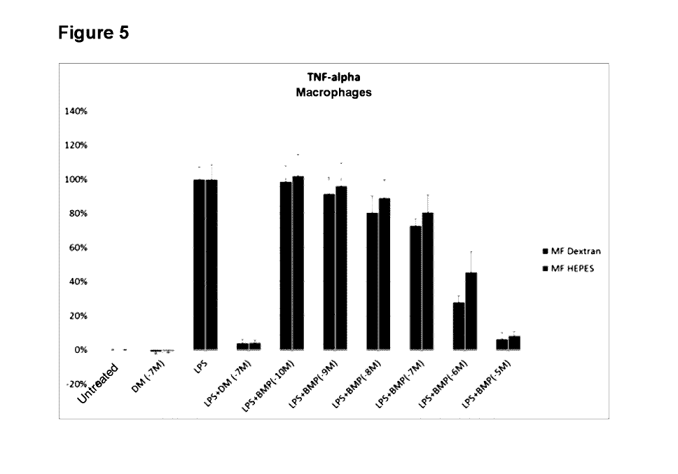

FIG. 5 shows the release of active ingredient from inorganic-organic hybrid compounds having an active ingredient anion and a fluorescent dye anion, using the example of [ZrO[BMP]²⁻_{0.9}[FMN]²⁻_{0.1}, the activity of BMP as anti-inflammatory being shown in lipopolysaccharide (LPS) stimulated peritoneal macrophages (MF) freshly isolated from mice, with an increasing concentration from 10⁻¹⁰ molar up to 10⁻⁵ molar, in comparison to dexamethasone (DM) (BMP: betamethasone phosphate; FMN: flavin mononucleotide).

Under physiological conditions, the active ingredient is released from the active ingredient anion very slowly, over a period of several hours to several days, and is able to develop its activity at the locus of action. This is shown in FIG. 4 for slow release in a laboratory suspension. The active ingredient anions here may be selected from a very broad range in terms of composition, and cover a wide range of possible clinical pictures. In addition to the release of an active ingredient, the inorganic-organic hybrid compounds of the invention can be located by the fluorescence of an additionally incorporated fluorescent dye anion. FIG. 3 shows that the distribution and location of the inorganic-organic hybrid compounds following intravenous administration can be depicted in the mouse model in vivo by means of noninvasive near-infrared imaging. FIG. 2 shows the uptake and accumulation of the inorganic-organic hybrid compounds in cells (macrophages). FIG. 5 shows the activity of betamethasone phosphate, which is released as active ingredient from the inorganic-organic hybrid compounds and has an anti-inflammatory activity in LPS-stimulated macrophages according to in vitro assays. In order to bring the nanoparticles in vivo to a specific locus of action, the inorganic-organic hybrid nanoparticles may additionally be functionalized with a peptide, antibody or oligonucleotide which is directed against a disease-specific target.

In summary the present invention has the following advantages over the prior art:
inorganic-organic hybrid compounds as an innovative materials concept for administration of active ingredients;
preferably the inorganic-organic hybrid compound contains both an active ingredient for therapy and a fluorescent dye for diagnostics;
the chemical composition is selectable within a broad range, specifically with inorganic metal cations selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or with a lanthanoid, and with an organic anion, comprising an active ingredient and optionally additionally a fluorescent dye, which carries in each case a phosphate, phosphonate, sulfate, sulfonate, carbonate or carboxylate group as functional group (and is therefore identified as active ingredient anion or fluorescent dye anion, respectively);
the inorganic-organic hybrid compound is recognizable through simple optical detection via the fluorescence of the fluorescent dye anion in cells, tissues or organs. Additionally possible is detection by X-ray absorption or magnetic measurements in the presence of heavy or magnetic inorganic cations (e.g., Ba²⁺, [ZrO]²⁺, [HfO]²⁺, Gd³⁺, La³⁺, Fe³⁺, Bi³⁺);
active ingredient anion and fluorescent dye anion are present in the inorganic-organic hybrid compound in molar amounts, the molar amount of active ingredient anion and fluorescent dye anion being in stoichiometric proportion to the inorganic cation, taking account of the respective ion charges.

Additionally the ratio of active ingredient anion to fluorescent dye anion may be varied;
the inorganic-organic hybrid compound is in general not crystalline (i.e., X-ray-amorphous). There is no need for the costly and inconvenient synthesis of crystalline materials and/or for the development of core/shell structures. This simplifies synthesis very greatly. Moreover, synthesis may take place by simple precipitation in aqueous phase, since the hybrid compounds are sparingly soluble in water. Hence the formation of seeds and the growth of seeds in water can also be readily controlled, allowing access very easily to particle diameters below 100 nm;
the inorganic-organic hybrid compound may contain a very wide variety of different active ingredient anions and can therefore be used for a very broad medical therapeutic territory (e.g., chronic inflammation/rheumatism/arthritis/multiple sclerosis, inflammation in general, tumor disorders, malaria, tuberculosis, angina pectoris, or coronary deposits). The active ingredient is released from the inorganic-organic hybrid compound under physiological conditions with a time delay, over a period of several hours to several days. By this route, a defined dose of active ingredient can be released over a prolonged time period and directly at the locus of action. Side-effects and the unwanted physiological degradation of active ingredient, in the blood, for example, can be lowered or avoided;
the inorganic-organic hybrid compound may also contain very different fluorescent dye anions. Excitation takes place typically in the spectral range of visible light, using appropriate lasers or light-emitting diodes (LEDs) which emit in the visible light or in the infrared range, respectively;
in addition to the active ingredient, the inorganic-organic hybrid compounds are notable for nonallergenic and/or nontoxic constituents, which are excreted in fully degraded form under physiological conditions;
in order to channel the inorganic-organic hybrid nanoparticles to a specific locus of action in vivo and to accumulate them there, they may additionally be functionalized with an antibody or peptide which is directed against a disease-specific target. In view of the water-based synthesis of the inorganic-organic hybrid compounds, this coupling as well with antibodies or similar molecules is particularly simple and gentle.

The compounds disclosed in the above-cited US 2011/0064775 are different in two key respects from the inorganic-organic hybrid compounds of the invention:
(i) The inorganic-organic hybrid compounds of the invention are composed of a cation and at least one active ingredient anion. There is a homogeneous composition present. The compounds described in US 2011/0064775 A1 are heterogeneous, meaning that they contain two substances which are clearly separable from one another (cf. FIG. 2 of US 2011/0064775 A1). US 2011/0064775 A1 describes the inclusion of nanoparticles and of molecules in an organometallic coordination polymer (Zn-Bix). This coordination polymer (Zn-Bix) is not actively fluorescent and is not an active ingredient. The matrix (Zn-Bix) is therefore functionless in terms of the function of the particles (fluorescence, active ingredient). In contrast to the inorganic-organic hybrid compounds of the invention, the function is carried by the included molecules or nanoparticles. Accordingly the total amount of active molecules for the system as a whole is limited.
(ii) The compounds described in US 2011/0064775 A1 are soluble in water and must therefore be prepared in absolute, anhydrous ethanol. Conversely, the hybrid compounds of the invention are sparingly soluble in water. This is essential in order for the hybrid compounds of the invention to be able to be prepared in water and/or to be used in aqueous systems (e.g. medicine, i.e., in cell fluid or blood).

The compounds disclosed in the above-cited U.S. Pat. No. 8,779,175 B2 likewise differ fundamentally from the inorganic-organic hybrid compounds of the invention:
(i) The compounds described in U.S. Pat. No. 8,779,175 B2 are soluble in water and must therefore be prepared, for example, in absolute, anhydrous methanol (e.g., magnesium topiramate, column 21, line 41ff.). Where synthesis is carried out in water, any excess starting materials can be separated off on the basis of their low solubility in water. The coordination complex, as target compound, is soluble in water, in contrast, and as a solid can be obtained only by distillative removal of the water (on a rotary evaporator) (an example being calcium topiramate). Conversely, the hybrid compounds of the invention are sparingly soluble in water. This is essential in order to be able to prepare the hybrid compounds of the invention in water and to use them in aqueous systems (e.g. medicine, i.e., in cell fluid or blood).
(ii) The substances obtained in U.S. Pat. No. 8,779,175 B2 are unstructured solids, not nanomaterials. This significantly limits their use in thin layers (no optical transparency) and in medicine (no intravenous administration).

In contrast to the metal hydroxide compounds disclosed in DE 20 2006 024 289, the inorganic-organic hybrid compounds of the invention can comprise the active ingredient molecule as single and exclusive anion. This is chemically not possible with layer-forming metal hydroxides. The framework (layer-forming hydroxide) is functionless with regard to the function of the substance (fluorescence, active ingredient). The function, in contrast to the inorganic-organic hybrid compounds of the invention, is carried by the attached molecules or nanoparticles. Hence the overall amount of active molecules for the system as a whole is limited.

Relative to the prior-art documents above, the amount of fluorescent anion or active ingredient anion, respectively, in the inorganic-organic hybrid compounds of the invention can be equimolar with the respective cation, this being an advantage because it allows extremely high levels of active ingredient and fluorescent dye, respectively, to be attained ([ZrO][BMP] for example contains 81 wt % of the active ingredient BMP). Similar levels apply in respect of [ZrO]²⁺[FdUMP]²⁻, for example. The inorganic-organic hybrid compounds of the invention can be prepared in water and are sparingly soluble in water. If the compounds are not sparingly soluble in water, nanoparticles cannot be prepared or stored in the presence of water (aqueous dissolution). Use in cells or in blood is not possible if the compounds are readily soluble under aqueous conditions.

The present invention is elucidated further by the nonlimiting examples hereafter.

### EXAMPLES

### Working Example 1

### [ZrO][BMP]_{0.9}[FMN]_{0.1}

The inorganic-organic hybrid compound ZrO(BMP)_{0.9}(FMN)_{0.1} is prepared by mixing of two solutions. Solution 1 contains ZrOCl₂.8H₂O (5 mg) in demineralized water (2.5 ml). Solution 2 contains sodium riboflavin 5′-monophosphate dihydrate (2.4 mg) and sodium betamethasone phosphate (21.6 mg) in demineralized water (25 ml). Solution 2 is heated to 50° C. and stirred vigorously (about 1000 rpm). Then solution 1 was injected rapidly with a syringe, with intensive stirring. After two minutes of stirring, the yellow-colored solid is separated off by centrifugation (15 min at 22 500 rev min⁻¹). The nanoparticles are resuspended twice in demineralized water (25 ml) and centrifuged again to remove all of the remaining salts. Finally, stable suspensions are obtained by resuspending the nanoparticles in HEPES buffer (12 ml, 30 mmol/l, pH=7.4). Alternatively the centrifugate is resuspended in demineralized water (3.1 ml). Added subsequently dropwise to this suspension, with stirring, is a solution of dextran 40 (3 ml, 1.6 mg/ml H₂O). In all cases the demineralized water used is rendered dust-free and germ-free before use, beforehand, by means of a sterile syringe filter (PA, 0.20 μm). This gives the inorganic-organic hybrid compound [ZrO][BMP]_{0.9}[FMN]_{0.1}, which comprises [ZrO]²⁺ as inorganic cation, the active ingredient anion [BMP]²⁻, and the fluorescent dye anion [FMN]²⁻, in the form of amorphous nanoparticles having a diameter of about 60 nm.

### Further Working Examples

In analogy to working example 1, instead of betamethasone phosphate [BMP]²⁻ as active ingredient anion, it is possible to use other active ingredient anions, and also, instead of flavin mononucleotide [FMN]²⁻ as fluorescent dye anion, it is possible to use other fluorescent dye anions in the inorganic-organic hybrid compounds of the invention, as shown by way of example below; cf. compounds 1 to 45. Additionally it is possible to select cations other than [ZrO]²⁺, such as Mg²⁺, Ca²⁺, Sr²⁺, Ba²⁺, Zn²⁺, [ZrO]²⁺, [HfO]²⁺, Sc³⁺, Y³⁺, Gd³⁺, La³⁺, Fe³⁺ or Bi³⁺, with inorganic cation and organic anion being used to synthesize a hybrid compound which is sparingly soluble in water.

Indicated below are salts or the free acid of the respective functional anion, which can alternatively both be used. The specified form corresponds to a customary, commercially available form of the functional organic anion in question, as may be used as a starting substance for the synthesis of inorganic-organic hybrid nanoparticles of the invention.

### 1. Acetaminophen Phosphate

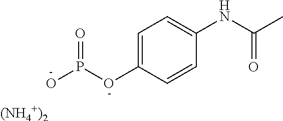

### 2. Betamethasone Phosphate

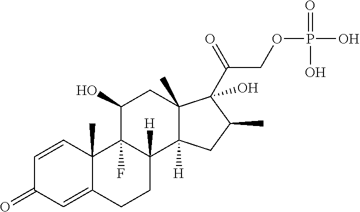

### 3. Dexamethasone Phosphate

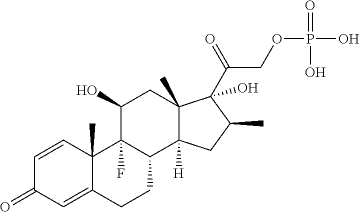

### 4. Methylprednisolone Phosphate

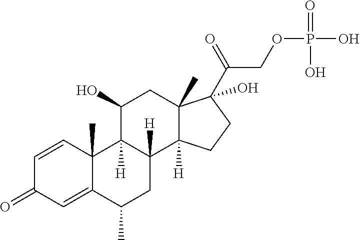

### 5. Triamcinolone Phosphate

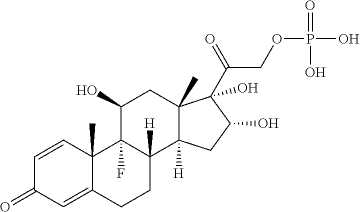

### 6. Estrone Phosphate

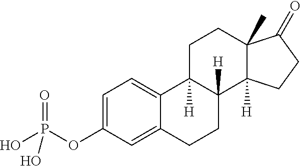

### 7. Testosterone Phosphate

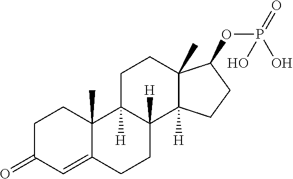

### 8. Estramustine Phosphate

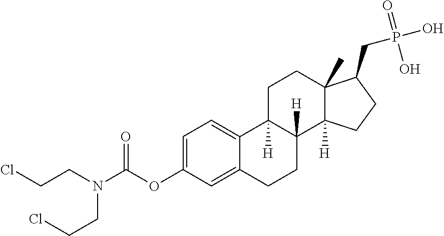

### 9. Codeine Phosphate

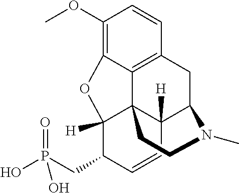

### 10. Clindamycin Phosphate

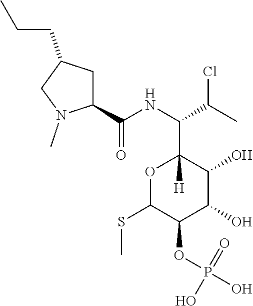

### 11. Thiamine PyroPhosphate

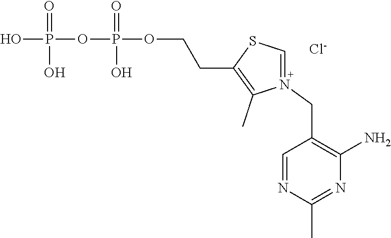

### 12. Thiamine Phosphate

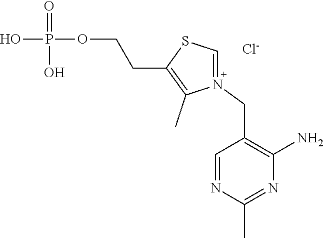

### 13. Aracytidine Monophosphate (ara-CMP)

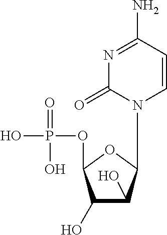

### 14. Cyclic 3′,5′-adenosine monophosphate

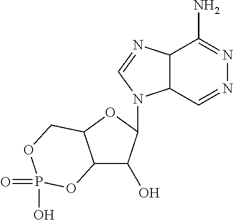 [SMILES: NC1=NN=CC2C1N=CN2C1OC2COP(=O)(O)OC2C1O]

### 15. Vidaribine Phosphate

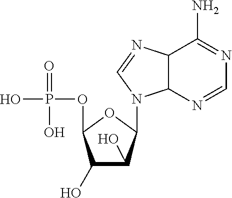

### 16. 9-[9-(Phosphonomethoxy)ethoxy]adenine

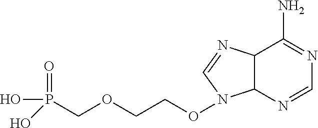 [SMILES: NC1=NC=NC2C1N=CN2OCCOCP(=O)(O)O]

### 17. Fospropofol (Lusedra®)

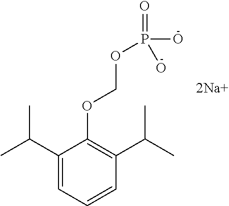 [SMILES: CC(C)c1cccc(C(C)C)c1OCOP(=O)(O)O]

### 18. Fosphenytoin

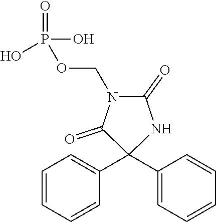 [SMILES: O=C1NC(c2ccccc2)(c2ccccc2)C(=O)N1COP(=O)(O)O]

### 19. Phosphoryloxymethyloxymethylphenytoin

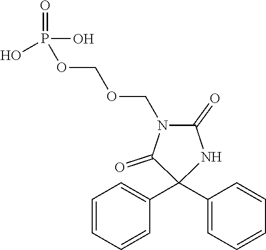 [SMILES: O=C1NC(c2ccccc2)(c2ccccc2)C(=O)N1COCOP(=O)(O)O]

### 20. Phosphoryloxymethylphenylbutazone

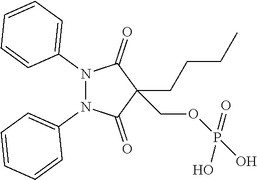 [SMILES: CCCCC1(COP(=O)(O)O)C(=O)N(c2ccccc2)N(c2ccccc2)C1=O]

### 21. Phosphoryloxvmethyloxymethylphenylbutazone

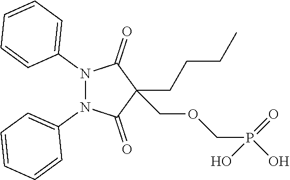 [SMILES: CCCCC1(COCP(=O)(O)O)C(=O)N(c2ccccc2)N(c2ccccc2)C1=O]

### 22. Phosphoryloxymethylphenindione

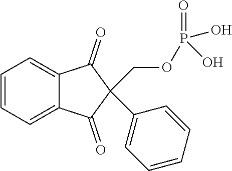 [SMILES: O=C1c2ccccc2C(=O)C1(COP(=O)(O)O)c1ccccc1]

### 23. Phosphoryloxymethyloxymethylphenindione

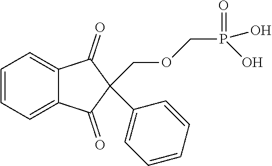 [SMILES: O=C1c2ccccc2C(=O)C1(COCP(=O)(O)O)c1ccccc1]

### 24. N-Phosphonooxymethylcinnarizine

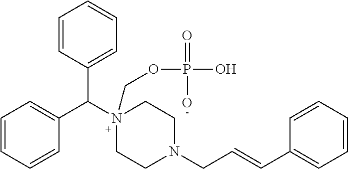

### 25. N-Phosphonooxymethylloxapine

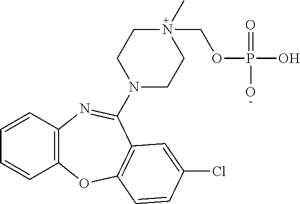

### 26. N-Phosphonooxymethylamiodarone

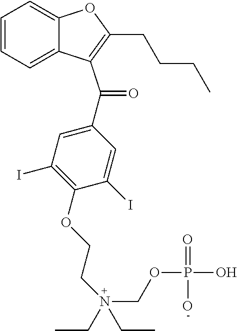

### 27. Alendronate

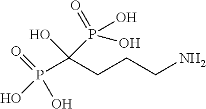 [SMILES: NCCCC(O)(P(=O)(O)O)P(=O)(O)O]

### 28. Resorufin Phosphate

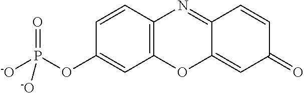

### 29. Brilliant Black

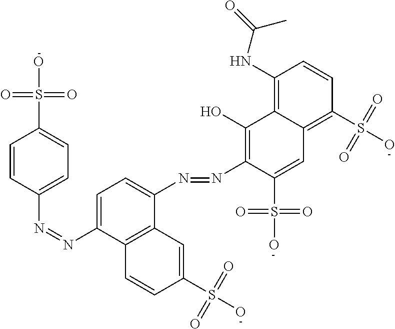 [SMILES: CC(=O)Nc1ccc(S(=O)(=O)O)c2cc(S(=O)(=O)O)c(/N=N/c3ccc(/N=N\c4ccc(S(=O)(=O)O)cc4)c4ccc(S(=O)(=O)O)cc34)c(O)c12]

### 30. 3-Phenylumbelliferone phosphate

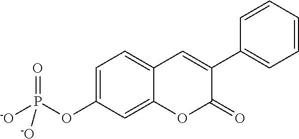

### 31. 3-O-Methylfluorescein phosphate

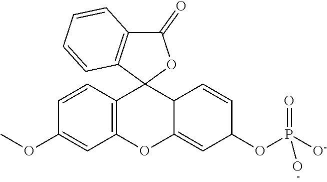 [SMILES: COc1ccc2c(c1)OC1=CC(OP(=O)([O-])O)C=CC1C21OC(=O)c2ccccc21]

### 32. Amaranth/Acid Red 27/Azorubin S

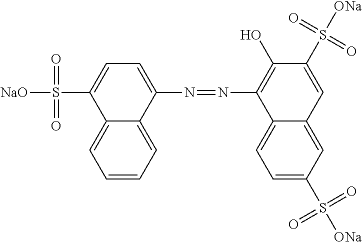

### 33. Cibacron Brilliant Red 3B-A/Reactive Red 4

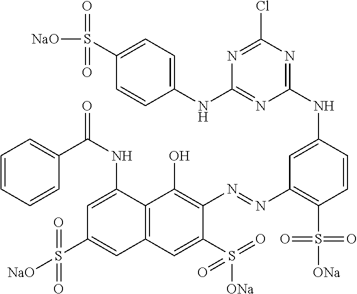

### 34. Acid Anthracene Red G

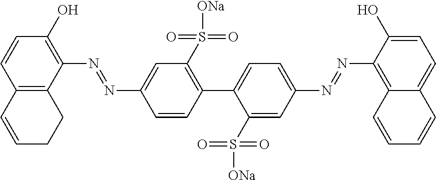

### 35. Nuclear Fast Red/Calcium Red

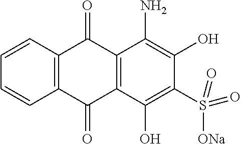

### 36. Potassium Canrenoate

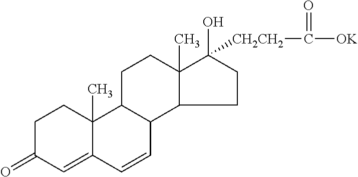

### 37. Doxycyclin Hydrate/Doxycyclin Hydrochloride Hemiethanolate Hemihydrate

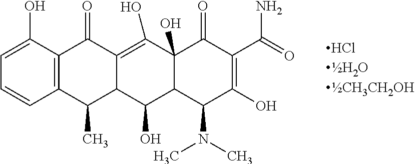 [SMILES: C[C@H]1c2cccc(O)c2C(=O)C2=C(O)[C@]3(O)C(=O)C(C(N)=O)=C(O)[C@@H](N(C)C)C3[C@@H](O)C21]

### 38. Calcein/Fluorexone/Fluorescein-bis(methyliminodiacetic Acid)

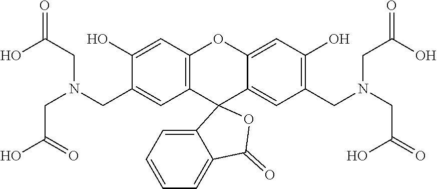 [SMILES: O=C(O)CN(CC(=O)O)Cc1cc2c(cc1O)Oc1cc(O)c(CN(CC(=O)O)CC(=O)O)cc1C21OC(=O)c2ccccc21]

### 39. Nitrazine Yellow

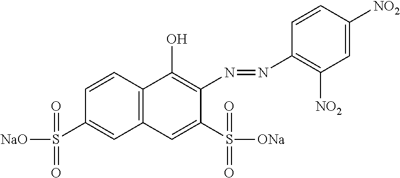

### 40. ABTS/2,2′-azino-bis(3-ethylbenzothiazoline-6-sulfonic acid) diammonium Salt

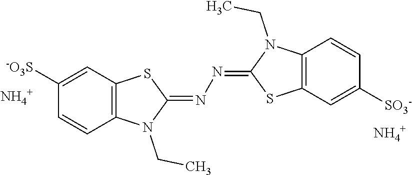

### 41. Doxorubicin Hydrochloride

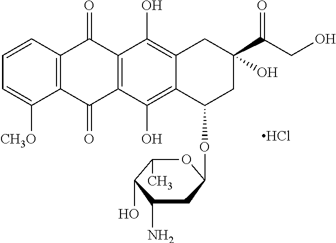

### 42. ANSA Magnesium Salt/8-anilino-1-naphthalenesulfonic Acid Hemimagnesium Salt Hydrate

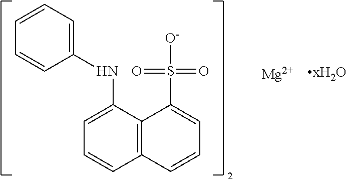

### 43. Indocyanine Green/Cardio Green

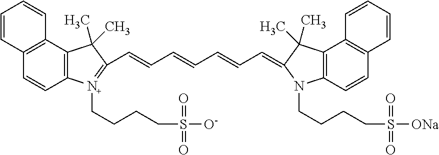

### 44. Lucifer Yellow CH Dilithium Salt

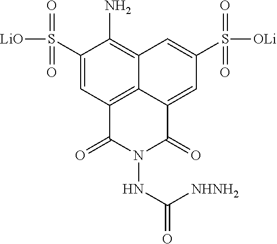

### 45. Fluorescent Red 633

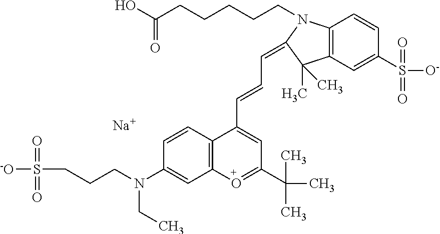

### 46. Aluminum(III)-phthalocyanine chloride tetrasulfonic Acid

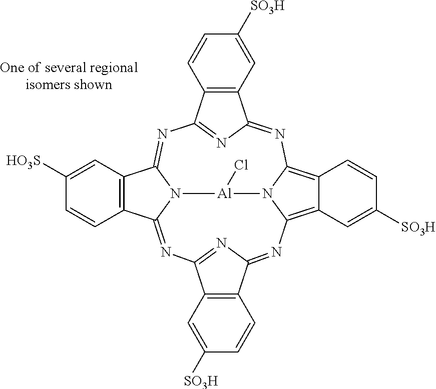

### 47. Aztreonam

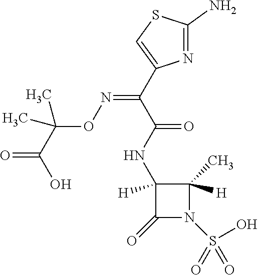 [SMILES: C[C@H]1[C@H](NC(=O)/C(=N\OC(C)(C)C(=O)O)c2csc(N)n2)C(=O)N1S(=O)(=O)O]

### 48. Tiaemonam

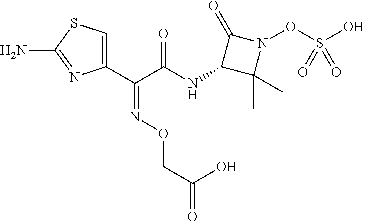 [SMILES: CC1(C)[C@H](NC(=O)/C(=N\OCC(=O)O)c2csc(N)n2)C(=O)N1OS(=O)(=O)O]

### 49. D-Glucosamine 6-sulfate

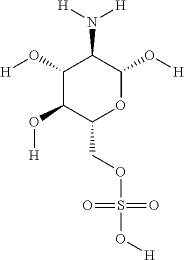 [SMILES: N[C@@H]1[C@@H](O)[C@H](O)[C@@H](COS(=O)(=O)O)O[C@H]1O]

### 50. Colistin Methanesulfate

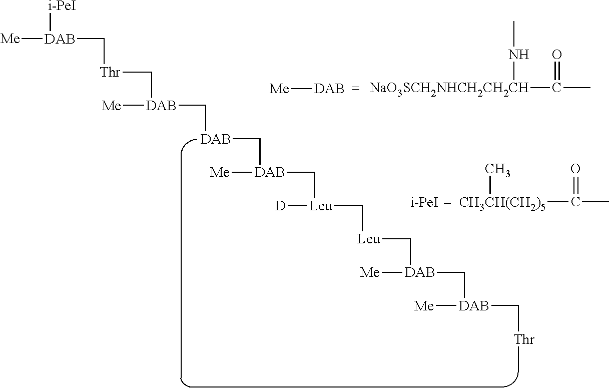

### 51. Cefsulodine

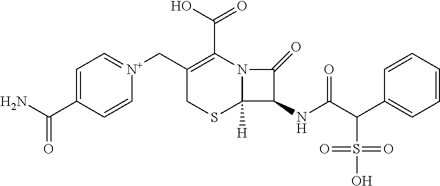 [SMILES: NC(=O)c1cc[n+](CC2=C(C(=O)O)N3C(=O)[C@@H](NC(=O)C(c4ccccc4)S(=O)(=O)O)[C@H]3SC2)cc1]

### 52. Fosamprenavir

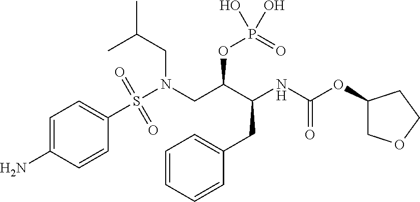 [SMILES: CC(C)CN(C[C@@H](OP(=O)(O)O)[C@H](Cc1ccccc1)NC(=O)O[C@H]1CCOC1)S(=O)(=O)c1ccc(N)cc1]

### 53. Tenofovir

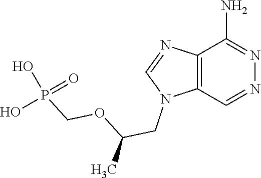 [SMILES: C[C@H](Cn1cnc2c(N)nncc21)OCP(=O)(O)O]

### 54. Adefovir

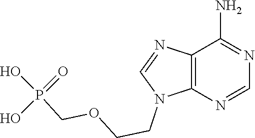 [SMILES: Nc1ncnc2c1ncn2CCOCP(=O)(O)O]

### 55. Combretastatin A-4 Phosphate

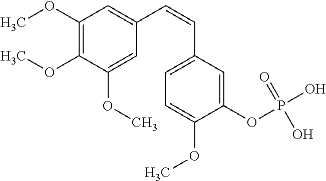 [SMILES: COc1ccc(/C=C\c2cc(OC)c(OC)c(OC)c2)cc1OP(=O)(O)O]

### 56. Folic Acid

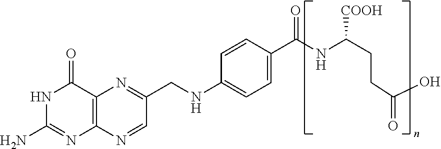

### 57. Fosphenytoin

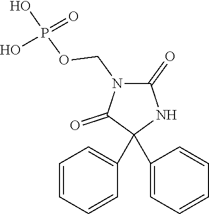 [SMILES: O=C1NC(c2ccccc2)(c2ccccc2)C(=O)N1COP(=O)(O)O]

### 58. 2-Mercaptoethanesulfonate/mesna

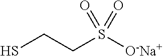

### 59. Fosfomycin

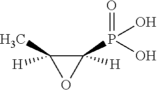

### 60. Glyphosate

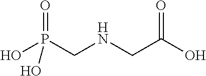

### 61. Glufosinate

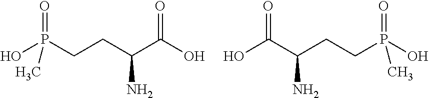

### 62. Zolendronate

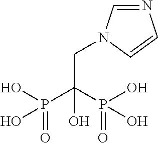 [SMILES: O=P(O)(O)C(O)(Cn1ccnc1)P(=O)(O)O]

### 63. Aminotrimethylenephosphonic Acid

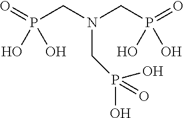 [SMILES: O=P(O)(O)CN(CP(=O)(O)O)CP(=O)(O)O]

### 64. Diethylenetriaminepenta(methylenephosphonic Acid)

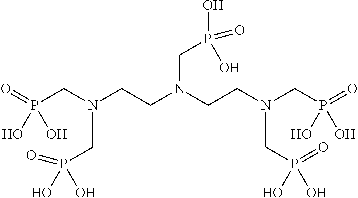 [SMILES: O=P(O)(O)CN(CCN(CP(=O)(O)O)CP(=O)(O)O)CCN(CP(=O)(O)O)CP(=O)(O)O]

### 65. Ethylenediaminetetra(methylenephosphonic Acid)

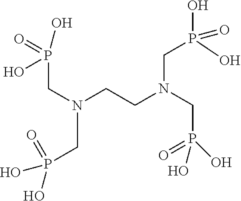 [SMILES: O=P(O)(O)CN(CCN(CP(=O)(O)O)CP(=O)(O)O)CP(=O)(O)O]

### 66. Fosbretabulin

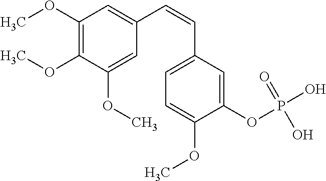 [SMILES: COc1ccc(/C=C\c2cc(OC)c(OC)c(OC)c2)cc1OP(=O)(O)O]

### 67. α-Tocopherol Phosphate

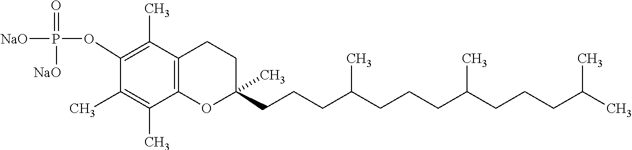

### 68. VAPOL Hydrogen Phosphate

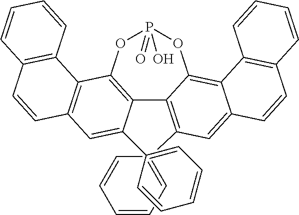

### 69. Pyridoxal 5′-phosphate 6-(2′-naphthylazo-6′-nitro-4′,8′-disulfonate)

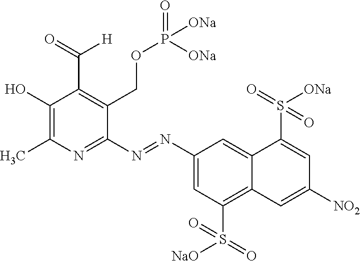

### 70. (11bR)-2,6-Di-9-phenanthrenyl-4-hydroxy-dinaphtho[2,1-d:1′,2′-f] [1,3,2]dioxaphosphepine 4-oxide

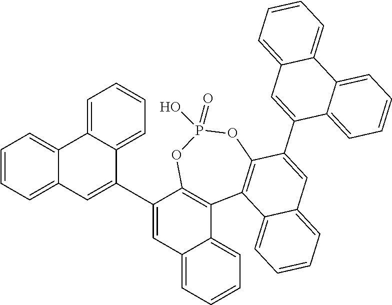 [SMILES: O=P1(O)Oc2c(-c3cc4ccccc4c4ccccc34)cc3ccccc3c2-c2c(c(-c3cc4ccccc4c4ccccc34)cc3ccccc23)O1]

### 71. 8-Bromo Cyclic Adenosine Diphosphate Ribose

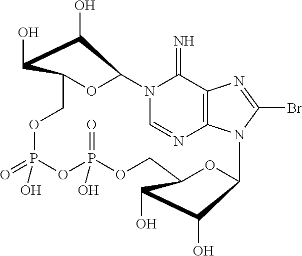 [SMILES: N=c1c2nc(Br)n3c2ncn1[C@H]1O[C@@H](COP(=O)(O)OP(=O)(O)OC[C@H]2O[C@@H]3[C@@H](O)C2O)C(O)[C@H]1O]

### 72. Phytic Acid

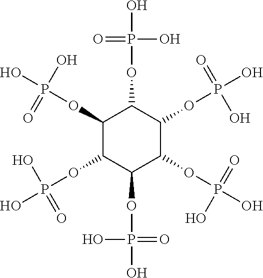

### 73. Trypan Blue

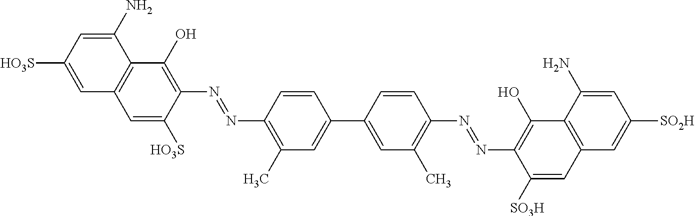

### 74. Glucose 6-phosphate and Other Phosphoric Esters of Sugars

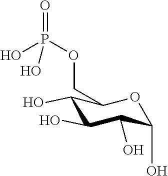

### 75. Naturally Occurring and Synthetic Nucleotides such as

Adenosine Monophosphate (AMP)

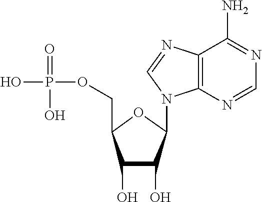

Adenosine Diphosphate (ADP)

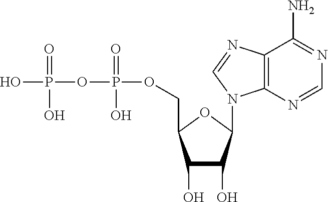

Adenosine Triphosphate (ATP)

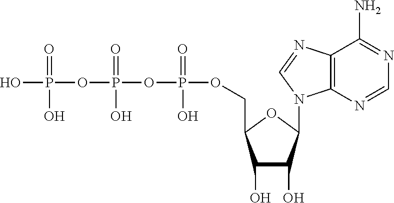

Guanosine Monophosphate (GMP)

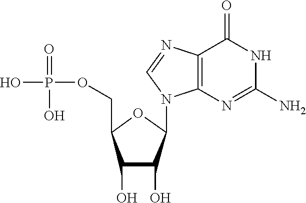

Guanosine Diphosphate (GDP)

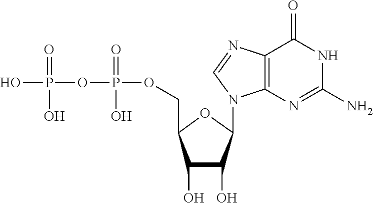

Guanosine Triphosphate (GTE)

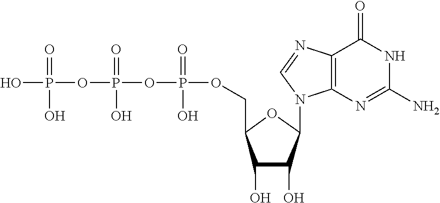

Cytidine Monophosphate (CMP)

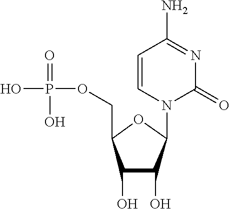

Cytidine Diphosphate (CDP)

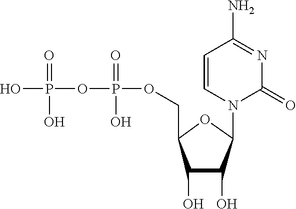

Cytidine Triphosphate (CTP)

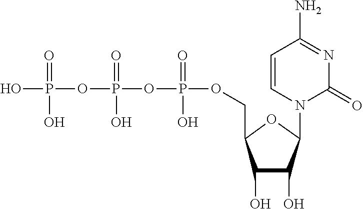

Uridine Monophosphate (UMP)

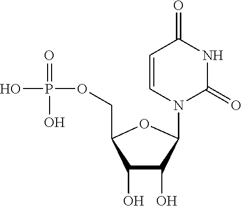

Uridine Diphosphate (UDP)

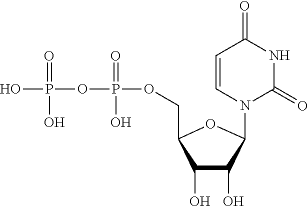

Uridine Triphosphate (UTP)

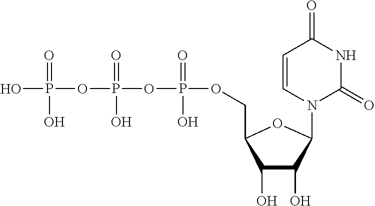

Deoxyadenosine Monophosphate (dAMP)

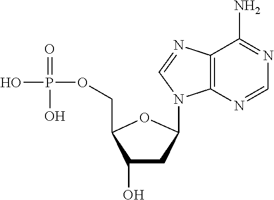 [SMILES: Nc1ncnc2c1ncn2[C@H]1C[C@@H](O)[C@@H](COP(=O)(O)O)O1]

Deoxyadenosine Diphosphate (dADP)

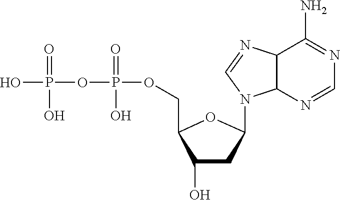 [SMILES: NC1=NC=NC2C1N=CN2[C@H]1C[C@@H](O)[C@@H](COP(=O)(O)OP(=O)(O)O)O1]

Deoxyguanosine Triphosphate (dATP)

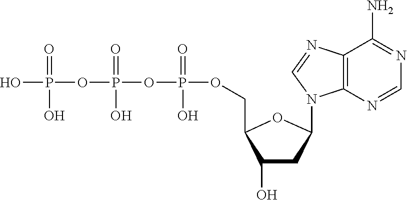 [SMILES: Nc1ncnc2c1ncn2[C@H]1C[C@@H](O)[C@@H](COP(=O)(O)OP(=O)(O)OP(=O)(O)O)O1]

Deoxyguanosine Monophosphate (dGMP)

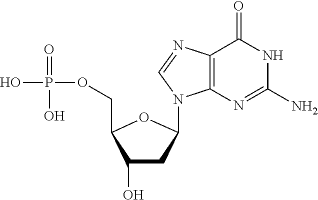 [SMILES: Nc1nc2c(ncn2[C@H]2C[C@@H](O)[C@@H](COP(=O)(O)O)O2)c(=O)[nH]1]

Deoxyguanosine Diphosphate (dGDP)

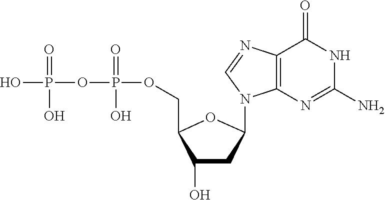 [SMILES: Nc1nc2c(ncn2[C@H]2C[C@@H](O)[C@@H](COP(=O)(O)OP(=O)(O)O)O2)c(=O)[nH]1]

Deoxyguanosine Triphosphate (dGTP)

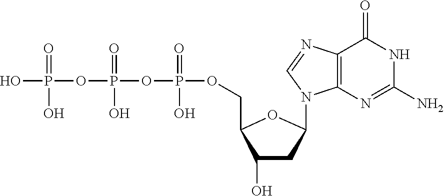 [SMILES: Nc1nc2c(ncn2[C@H]2C[C@@H](O)[C@@H](COP(=O)(O)OP(=O)(O)OP(=O)(O)O)O2)c(=O)[nH]1]

Deoxycytidine Monophosphate (dCMP)

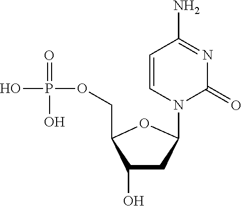 [SMILES: Nc1ccn([C@H]2C[C@@H](O)[C@@H](COP(=O)(O)O)O2)c(=O)n1]

Deoxycytidine Diphosphate (dCDP)

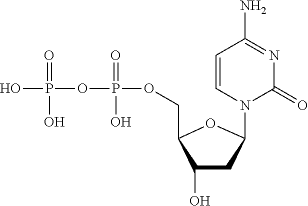 [SMILES: Nc1ccn([C@H]2C[C@@H](O)[C@@H](COP(=O)(O)OP(=O)(O)O)O2)c(=O)n1]

Deoxycytidine Triphosphate (dCTP)

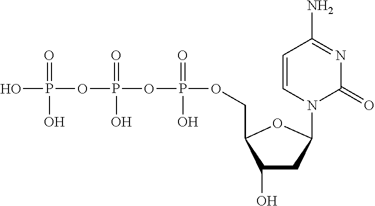 [SMILES: Nc1ccn([C@H]2C[C@@H](O)[C@@H](COP(=O)(O)OP(=O)(O)OP(=O)(O)O)O2)c(=O)n1]

Deoxythymidine Monophosphate (dTMP)

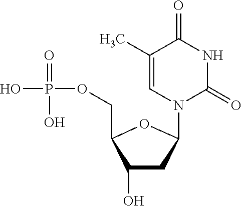 [SMILES: Cc1cn([C@H]2C[C@@H](O)[C@@H](COP(=O)(O)O)O2)c(=O)[nH]c1=O]

Deoxythymidine Diphosphate (dTDP)

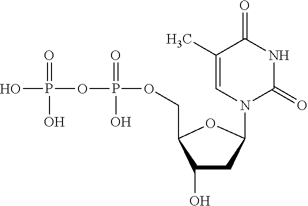 [SMILES: Cc1cn([C@H]2C[C@@H](O)[C@@H](COP(=O)(O)OP(=O)(O)O)O2)c(=O)[nH]c1=O]

Deoxythymidine Triphosphate (dTTP)

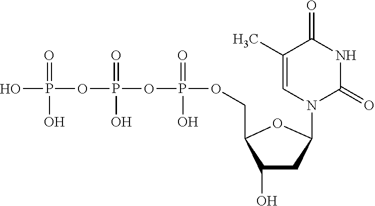 [SMILES: Cc1cn([C@H]2C[C@@H](O)[C@@H](COP(=O)(O)OP(=O)(O)OP(=O)(O)O)O2)c(=O)[nH]c1=O]

Additionally it is also possible to use luminescent-labeled nucleotides for the synthesis of luminescent hybrid compounds. Such nucleotides are available commercially, as for example from Life Technologies (under the ALEXA name) or Dyomics (under the DY name). The dyes are IR-fluorescent dyes. In so far as visible light is absorbed very quickly in the tissue, at a few micrometers of tissue thickness, IR emission is particularly advantageous in medicine in view of the low tissue penetration of IR light. These IR dyes are standard dyes for medical application. The luminescent-labeled nucleotides listed by way of example below all contain a phosphate group and can be incorporated readily into the hybrid compounds of the invention. Product names (e.g., ALEXA or DY), the excitation and emission wavelengths, and in some cases the empirical formula of the compounds are given below.

| Luminescent- | Molar | | |
|---|---|---|---|
| modified | mass in | | Exc/Em |
| nucleotide | g mol⁻¹ | Empirical formula | in nm |
| DY-630-dUTP | 1163.78 | C₄₈H₆₀N₅O₁₉P₃S * 4Li | 636/657 (in |
| | | | ethanol) |
| DY-631-dUTP | 1362.94 | C₅₄H₇₀N₆O₂₃P₃S₂ * 5Li | 637/658 (in |
| | | | ethanol) |
| DY-632-dUTP | 1462.96 | C₅₅H₇₁N₆O₂₆P₃S₃Li₆ | 637/657 (in |
| | | | ethanol) |
| DY-633-dUTP | 1263.80 | C₄₉H₆₁N₅O₂₂P₃S₂ * 5Li | 637/657 (in |
| | | | ethanol) |
| DY-634-dUTP | 1562.98 | C₅₆H₇₂N₆O₂₉P₃S₄Li₇ | 635/658 (in |
| | | | ethanol) |
| DY-635-dUTP | 1187.80 | C₅₀H₆₀N₅O₁₉P₃SLi₄ | 647/671 (in |
| | | | ethanol) |
| DY-636-dUTP | 1386.96 | C₅₆H₇₀N₆O₂₃P₃S₂ * 5Li | 645/671 (in |
| | | | ethanol) |
| DY-647P1-dUTP | 1221.72 | C₄₆H₅₅N₅O₂₂P₃S₂ * 5Li | 653/672 (in |
| | | | ethanol) |
| DY-648P1-dUTP | 1335.77 | C₄₈H₅₈N₅O₂₅P₃S₃ * 6Li | 653/672 (in |
| | | | ethanol) |
| DY-650-dUTP | 1215.86 | C₅₂H₆₄N₅O₁₉P₃SLi₄ | 653/674 (in |
| | | | ethanol) |
| DY-651-dUTP | 1415.01 | C₅₈H₇₄N₆O₂₃P₃S₂ * 5Li | 656/678 (in |
| | | | ethanol) |
| DY-652-dUTP | 1515.03 | C₅₉H₇₅N₆O₂₆P₃S₃Li₆ | 654/675 (in |
| | | | ethanol) |
| DY-677-dUTP | 1535.02 | C₆₁H₇₁N₆O₂₆P₃S₃Li₂ | 673/694 (in |
| | | | ethanol) |
| DY-678-dUTP | | | 674/694 (in |
| | | | ethanol) |
| DY-679P1-dUTP | | | 679/697 (in |
| | | | PBS) |
| DY-681-dUTP | 1362.94 | C₅₄H₇₀N₆O₂₃P₃S₂Li₅ | 691/708 (in |
| | | | ethanol) |
| DY-781-dUTP | 1388.98 | C₅₆H₇₂N₆O₂₃P₃S₂Li₅ | 783/800 (in |
| | | | ethanol) |

| Luminescent-modified nucleotide | Exc/Em in nm |
|---|---|
| Alexa Fluor 546-14-dUTP | 555/570 |
| Alexa Fluor 555-aha-dUTP | 555/570 |
| Alexa Fluor 555-aha-dCTP | 555/570 |
| Alexa Fluor 546-16-OBEA-dCTP | 555/570 |
| Alexa Fluor 546-14-UTP | 555/570 |
| Alexa Fluor 568-5-dUTP | 575/600 |
| Texas Red-12-dUTP | 595/610 |
| Alexa Fluor 594-5-dUTP | 590/615 |
| Alexa Fluor 647-aha-dUTP | 650/670 |
| Alexa Fluor 647-aha-dCTP | 650/670 |
| Alexa Fluor 647-12-OBEA-dCTP | 650/670 |

1. An inorganic-organic hybrid compound as ionic compound, composed of an inorganic metal cation selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or a lanthanoid, and of an organic active ingredient anion which comprises at least one phosphate, phosphonate, sulfate, sulfonate, carbonate or carboxylate group as functional group, the compound having a molar solubility of 10⁻² mol/l in water.

2. The inorganic-organic hybrid compound as claimed in claim 1, further comprising a fluorescent dye anion which carries a phosphate, phosphonate, sulfate, sulfonate, carbonate or carboxylate group as functional group.

3. The inorganic-organic hybrid compound as claimed in claim 1, which is in X-ray-amorphous form.

4. The inorganic-organic hybrid compound as claimed in claim 1, which has a particle diameter in the range from 1 to 100 nm.

5. The inorganic-organic hybrid compound as claimed in claim 1, which is doped with a lanthanoid selected from Ce, Pr, Nd, Sm, Eu, Gd, Tb, Dy, Ho, Er, Tm, Yb or Lu, with a transition metal selected from Cr, Mn, Cu, Zn, Y, Ag or Cd, with a main group element selected from Sn, Sb, Pb or Bi, or with a complex anion selected from [VO₄]³⁻, [MoO₄]³⁻ or [WO₄]³⁻.

6. The inorganic-organic hybrid compound as claimed in claim 1, whose active ingredient anion is directed against chronic inflammation, asthma, rheumatism, arthritis, multiple sclerosis, inflammation, tumor disorders, malaria, tuberculosis, angina pectoris or coronary deposits.

7. The inorganic-organic hybrid compound as claimed in claim 1, the active ingredient anion being derived from acetaminophen phosphate, betamethasone phosphate, dexamethasone phosphate, uridine monophosphate, 5′-fluoro-2′-deoxyuridine 5′-monophosphate, methylprednisolone phosphate, triamcinolone phosphate, estrone phosphate, testosterone phosphate, estramustine phosphate, codeine phosphate, clindamycin phosphate, thiamine pyrophosphate, thiamine phosphate; aracytidine monophosphate, cyclic 3′,5′-adenosine monophosphate, vidaribine phosphate, 9-[9-(phosphonomethoxy)ethoxy]adenine, fospropofol, fosphenytoin, phosphoryloxymethyloxymethylphenytoin, phosphoryloxymethylphenylbutazone, phosphoryloxymethyloxymethylphenylbutazone, phosphoryloxymethylphenindione, phosphoryloxymethyloxymethylphenindione, N-phosphonooxymethylcinnarizine, N-phosphonooxymethylloxapine, N-phosphonooxymethylamiodarone, alendronate, canrenoate, doxycyclin hydrate, doxorubicin hydrochloride, aztreonam, tigemonam, D-glucosamine 6-sulfate, colistin methane sulfate, cefsulodin, fosamprenavir, tenofovir, adefovir, combretastatin A-4 phosphate, folic acid, 2-mercaptoethansulfonate/mesna, fosfomycin, glyphosate, glufosinate, zolendronate, aminotrimethylenephosphonic acid, diethylenetriaminepenta(methylenephosphonic acid), ethylenediaminetetra(methylenephosphonic acid), fosbretabulin, α-tocopherol phosphate, VAPOL hydrogenphosphate, pyridoxal 5′-phosphate 6-2′-naphthylazo-6′-nitro-4′,8′-disulfonate), (11bR)-2,6-di-9-phenanthrenyl-4-hydroxydinaphtho [2,1-d:1′,2′-f] [1,3,2]dioxaphosphepine 4-oxide, 8-bromo cyclic adenosine diphosphate ribose, phytic acid, glucose 6-phosphate and other phosphoric esters of sugars, or naturally occurring and synthetic nucleotides including adenosine monophosphate (AMP), adenosine diphosphate (ADP), adenosine triphosphate (ATP), guanosine monophosphate (GMP), guanosine diphosphate (GDP), guanosine triphosphate (GTP), cytidine monophosphate (CMP), cytidine diphosphate (CDP), cytidine triphosphate (CTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxycytidine monophosphate (dCMP), deoxycytidine diphosphate (dCDP), deoxycytidine triphosphate (dCTP), deoxythymidine monophosphate (dTMP), deoxythymidine diphosphate (dTDP) or deoxythymidine triphosphate (dTTP).

8. The inorganic-organic hybrid compound as claimed in claim 1, which is further functionalized with an antibody, peptide or oligonucleotide.

9. The inorganic-organic hybrid compound as claimed in claim 1, the organic fluorescent dye anion being derived from fluorescent dyes selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluorophenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N,N′-difluoroboryl-1,9-dimethyl-5-(4-iodophenyl)dipyrrin, N,N′-difluoroboryl-1,9-dimethyl-5-[(4-(2-trimethylsilylethynyl), N,N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenyl-beta-octa(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, including tetra(o-amidophosphonatophenyl)porphyrin, and umbelliferone, where the organic fluorescent dyes which do not as such have a phosphate, phosphonate, sulfate, sulfonate, carbonate or carboxylate group have been modified with at least one of these functional groups.

10. A process for preparing an inorganic-organic hybrid compound as claimed in claim 1, comprising the steps of
(a) providing a solution of an organic active ingredient compound which has one or more functional groups selected from phosphate, phosphonate, sulfate, sulfonate, carbonate or carboxylate groups, the solution optionally further comprising at least one anion selected from phosphate, phosphonate, sulfate, sulfonate, carbonate or carboxylate,
(b) providing a solution of a soluble metal salt comprising metal cations selected from Mg²⁺, Ca²⁺, Sr²⁺, Ba²⁺, Zn²⁺, Zr⁴⁺, [ZrO]²⁺, [HfO]²⁺, Sc³⁺, Y³⁺, Gd³⁺, La³⁺, Fe³⁺, Bi³⁺ or a lanthanoid,
(c) combining the two solutions with stirring, to precipitate the hybrid compound, and
(d) isolating and/or purifying the precipitated hybrid compound.

11. The process as claimed in claim 10, where the solution provided in step (a) is further admixed with an organic fluorescent dye which has one or more functional groups selected from phosphate, phosphonate, sulfate, sulfonate, carbonate or carboxylate groups.
